(19) United States
(12) Patent Application Publication
(10) Pub. No.: US 2025/0188061 A1
(43) Pub. Date: Jun. 12, 2025

(54) NOVEL DEUTERATED PYRIMIDIN-2-YL SULFONAMIDE DERIVATIVES

(71) Applicant: Hoffmann-La Roche Inc., Little Falls, NJ (US)

(72) Inventors: Guido GALLEY, Rheinfelden (DE); Luca Claudio GOBBI, Buus (CH); Wolfgang GUBA, Muellheim (DE); Dmitry MAZUNIN, Grenzach-Wyhlen (DE); Emmanuel PINARD, Linsdorf (FR)

(73) Assignee: Hoffmann-La Roche Inc., Little Falls, NJ (US)

(21) Appl. No.: 19/059,580

(22) Filed: Feb. 21, 2025

Related U.S. Application Data
(63) Continuation of application No. PCT/EP2023/073189, filed on Aug. 24, 2023.

(30) Foreign Application Priority Data
Aug. 26, 2022 (EP) 22192252.9

(51) Int. Cl. C07D 403/12; A61K 31/506; C07D 471/04

(52) U.S. Cl. CPC C07D 403/12; A61K 31/506; C07D 471/04

(57) ABSTRACT

The invention relates to novel compounds having the general formula I

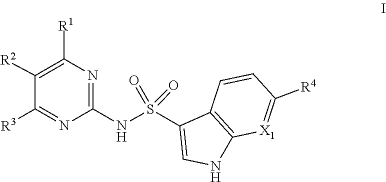

wherein the substituents R¹, R², R³, R⁴ and X₁ are as defined above, composition including the compounds and methods of using the compounds.

The present invention relates to organic compounds useful for therapy and/or prophylaxis in a mammal, and in particular to compounds that modulate GPR17 activity.

The present invention provides novel compounds of formula I

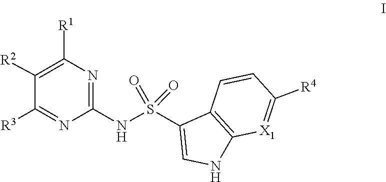

wherein
R¹ is H, alkoxy, or haloalkoxy;
R² is 1,1,2,2-tetradeuterio-2-fluoro-ethoxy, 1,1-dideuterio-2-fluoro-ethoxy, or 1,1-dideuterio-2,2-difluoro-ethyl;
R³ is alkoxy or haloalkoxy;
X₁ is CR⁵ or N;
R⁴ is H, halo, alkyl, or haloalkyl;
R⁵ is H or halo;
and pharmaceutically acceptable salts.

Furthermore, the invention includes all racemic mixtures, all their corresponding enantiomers and/or optical isomers.

### BACKGROUND OF THE INVENTION

Myelination is a process that occurs robustly during development and despite the abundant presence of oligodendrocyte precursor cells (OPCs) throughout the adult CNS, the transition to myelinating oligodendrocytes and the production of restorative myelin sheaths around denuded axons is impaired in chronic demyelinating diseases. During development, myelination proceeds in a very orderly manner, with OPCs, characterized by expression of markers such as neural/glial antigen 2 (NG2) and platelet-derived growth factor alpha (PDGFRα), differentiating into oligodendrocytes which lose NG2 and PDGFRα expression and gain the expression of markers such as myelin basic protein (MBP) and myelin oligodendrocyte glycoprotein (MOG). The production of myelin by oligodendrocytes is a very tightly regulated process and in the CNS, this can be controlled by interactions with axons, well-understood in the peripheral but not in the central nervous system (Macklin, W. B. (2010). Sci. Signal. 3, pe32-pe32. “The myelin brake: When Enough Is Enough”). Myelination can also be controlled by internal brakes within oligodendrocytes themselves, through the transcription factor EB (TFEB)-PUMA axis or through GPR17 antagonism (Chen, Y., et al. (2009). Nat Neurosci 12, 1398-1406, “The oligodendrocyte-specific G protein-coupled receptor GPR17 is a cell-intrinsic timer of myelination”) (Sun, L. O., et al. (2018). Cell 175, 1811-1826.e21, “Spatiotemporal Control of CNS Myelination by Oligodendrocyte Programmed Cell Death through the TFEB-PUMA Axis”). Myelin serves not only to protect axons and facilitate neuronal transmission, but oligodendrocytes have also been shown to play an important role in metabolism of axons as well as in maintaining the electrolyte balance around axons (Schirmer, L., et al. (2014). Ann Neurol 75, 810-828. “Differential loss of KIR4.1 immunoreactivity in multiple sclerosis lesions”) (Simons, M., and Nave, K.-A. (2015). Cold Spring Harb Perspect Biol. 22, “Oligodendrocytes: Myelination and Axonal Support”).

GPR17 is a Class A orphan G protein-coupled receptor (GPCR). GPCRs are 7 domain transmembrane proteins that couple extracellular ligands with intracellular signaling via their intracellular association with small, heterotrimeric G-protein complexes consisting of G_{α}, G_{β}, G_{γ} subunits. It is the coupling of the GPCR to the G_{α} subunit that confers results in downstream intracellular signaling pathways. GPR17 is known to be coupled directly to G_{αi/o}, which leads to inhibition of adenylate cyclase activity, resulting in a reduction in cyclic AMP production (cAMP). GPR17 has also been shown to couple to G_{q/11}, that targets phospholipase C. Activation of phospholipase C leads to the cleavage of phosphatidylinositol 4,5-bisphosphate which produces inositol triphosphate (IP₃) and diacylglycerol (DAG). IP₃ consequently binds to the IP₃ receptor on the endoplasmic reticulum and causes an increase in intracellular calcium levels (Hanlon, C. D., and Andrew, D. J. (2015). J Cell Sci. 128, 3533-3542, “Outside-in signaling-a brief review of GPCR signaling with a focus on the Drosophila GPCR family”) (Inoue, A., et al. (2019), Cell 177, 1933-1947.e25, “Illuminating G-Protein-Coupling Selectivity of GPCRs”).

The role of GPR17 in myelination was first identified in a screen of the optic nerves of Olig1 knockout mice to identify genes regulating myelination. GPR17 expression was found to be expressed only in the myelinating cells of the CNS and absent from the Schwann cells, the peripheral nervous system's myelinating cells. The expression of GPR17 was found to be exclusively expressed in the oligodendrocyte lineage cells and was downregulated in myelinating oligodendrocyte (Chen. Y., et al. (2009)). Specifically. GPR17 expression is found to be present at low levels early on in the OPC and increases in the pre-myelinating oligodendrocyte before the expression is downregulated in the mature. myelinating oligodendrocyte (Boda, E., et al. (2011). Glia 59. 1958-1973, “The GPR17 receptor in NG2 expressing cells: Focus on in vivocell maturation and participation in acute trauma and chronic damage”) (Dziedzic. A., et al. (2020). Int. J. Mol. Sci. 21. 1852. “The gpr17 receptor—a promising goal for therapy and a potential marker of the neurodegenerative process in multiple sclerosis”) (Fumagalli, M. et al. (2011). J Biol Chem 286. 10593-10604. “Phenotypic changes, signaling pathway, and functional correlates of GPR17-expressing neural precursor cells during oligodendrocyte differentiation”). GPR17 knockout animals were shown to exhibit precocious myelination throughout the CNS and conversely, transgenic mice overexpressing GPR17 in oligodendrocytes with the CNP-Cre (2′, 3′-cyclic-nucleotide 3′-phosphodiesterase) promoter exhibited myelinogenesis defects, in line with what is to be expected of a cell-intrinsic brake on the myelination process (Chen, Y., et al. (2009)). Furthermore, loss of GPR17 enhances remyelination following demyelination with lysophosphatidylcholine-induced demyelination (Lu, C., Dong, et al. (2018), Sci. Rep. 8, 4502, “G-Protein-Coupled Receptor Gpr17 Regulates Oligodendrocyte Differentiation in Response to Lysolecithin-Induced Demyelination”). As such, antagonism of GPR17 that promotes the differentiation of oligodendrocyte lineage cells into mature, myelinating oligodendrocytes would lead to increase in myelination following demyelination.

Multiple sclerosis (MS) is a chronic neurodegenerative disease that is characterized by the loss of myelin, the protective fatty lipid layer surrounding axons, in the central nervous system (CNS). Prevention of myelin loss or remyelination of denuded axons is thought to prevent axonal degeneration and thus prevent progression of the disease (Franklin. R. J. (2002), Nat Rev Neurosci 3, 705-714, “Why does remyelination fail in multiple sclerosis?”). Due to the restorative impact that myelin repair has on the central nervous system, such a treatment will benefit all types of MS namely relapse-remitting, secondary progressive, primary progressive and progressive relapsing MS. Reparation of lost myelin will alleviate neurological symptoms associated with MS due to the neuroprotective effect of preserving axons.

Due to the essential role that myelination plays in functioning of the nervous system, facilitating OPC to oligodendrocyte differentiation has the potential to impact multiple diseases where white matter defects/irregularities due to either loss of myelinating oligodendrocytes or hampered differentiation of OPCs to oligodendrocytes have been observed, due to the disease itself or inflammation. This is in addition to the diseases where GPR17 expression itself is altered.

The diseases that GPR17 antagonism can be thus used to yield a positive disease outcome include, but are not limited to:

Direct damage to myelin sheaths:
Metabolic conditions that lead to destruction of central myelin such as central pontine myelinolysis, extra-pontine myelinolysis due to overly-rapid correction of hyponatremia in conditions for instance, but not limited to, alcoholism, liver disease, immunosuppression after transplantation
Carbon monoxide poisoning where oligodendrocyte dysfunction and failure to regenerate has been reported in the deep white matter layers of the brain
Nutritional deficiency that results in myelin loss or failure to properly generate myelin during development
Virus-induced demyelination

Primary demyelinating disorders
Multiple Sclerosis (relapse-remitting, secondary progressive, primary progressive and progressive relapsing MS)
Acute and multiphasic disseminated encephalomyelitis
Neuromyelitis optica spectrum disorders including optic neuritis
Transverse myelitis
Leukodystrophies such as adrenoleukodystrophy, adrenomyeloneuropathy and other inherited leukodystrophies that result in myelin loss

CNS disorders with associated myelin loss:
Alzheimer's Disease
Schizophrenia
Parkinson's Disease
Huntington's disease
Amyotrophic lateral
Ischemia due to stroke

Other diseases:
Inflammation in the CNS for instance following encephalitis, primary angiitis, meningitis

The compounds of formula I bind to and modulates GPR17 activity.

The compounds of formula I are therefore particularly useful in the treatment of diseases related to GPR17 antagonism.

The compounds of formula I are particularly useful in the treatment or prophylaxis of multiple sclerosis (MS), conditions related to direct damage to myelin sheaths such as carbon monoxide poisoning or virus induced demyelination, primary demyelinating disorders such as acute and multiphasic disseminated encephalomyelitis, and other CNS disorders associated with myelin loss such as Alzheimer's disease, schizophrenia, Parkinson's disease and Huntington's disease.

### SUMMARY OF THE INVENTION

The present invention provides novel compounds of formula I

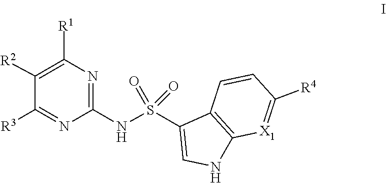

wherein
R¹ is H, alkoxy, or haloalkoxy;
R² is 1,1,2,2-tetradeuterio-2-fluoro-ethoxy, 1,1-dideuterio-2-fluoro-ethoxy, or 1,1-dideuterio-2,2-difluoro-ethyl;
R³ is alkoxy or haloalkoxy;
X₁ is CR⁵ or N;
R⁴ is H, halo, alkyl, or haloalkyl;
R⁵ is H or halo;
and pharmaceutically acceptable salts.

The term “alkyl” denotes a monovalent linear or branched saturated hydrocarbon group of 1 to 6 carbon atoms. In some embodiments, if not otherwise described, alkyl comprises 1 to 6 carbon atoms (C₁₋₆-alkyl), or 1 to 4 carbon atoms (C₁₋₄-alkyl). Examples of C₁₋₆-alkyl include methyl, ethyl, propyl, isopropyl, n-butyl, iso-butyl, sec-butyl, tert-butyl and pentyl. Particular alkyl groups include methyl, ethyl and propyl. When an alkyl residue having a specific number of carbons is named, all geometric isomers having that number of carbons may be encompassed. Thus, for example, “butyl” can include n-butyl, sec-butyl, isobutyl and t-butyl, and “propyl” can include n-propyl and isopropyl. Particular example of alkyl is methyl.

The term “alkoxy” denotes a group of the formula —O—R′, wherein R′ is a C₁₋₆-alkyl group. Examples of C₁₋₆-alkoxy groups include methoxy, ethoxy, n-propoxy, isopropoxy, n-butoxy, isobutoxy and tert-butoxy. Particular example is methoxy.

The term “halogen”, “halide” and “halo” are used interchangeably herein and denote fluoro, chloro, bromo or iodo. Particular halogens are fluoro, chloro and bromo.

The term “haloalkyl” denotes a C₁₋₆-alkyl group wherein at least one of the hydrogen atoms of the C₁₋₆-alkyl group has been replaced by the same or different halogen atoms.

The term “haloalkoxy” denotes a C₁₋₆-alkoxy group wherein at least one of the hydrogen atoms of the C₁₋₆-alkoxy group has been replaced by the same or different halogen atoms.

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition these salts may be prepared from addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine. N-ethylpiperidine, piperidine, polyamine resins. The compound of formula I can also be present in the form of zwitterions. Particularly preferred pharmaceutically acceptable salts of compounds of formula I are the salts formed with formic acid and the salts formed with hydrochloric acid yielding a hydrochloride, dihydrochloride or trihydrochloride salt.

The abbreviation uM means microMolar and is equivalent to the symbol μM.

The abbreviation uL means microliter and is equivalent to the symbol μL.

The abbreviation ug means microgram and is equivalent to the symbol μg.

The compounds of formula I can contain several asymmetric centers and can be present in the form of optically pure enantiomers, mixtures of enantiomers such as, for example, racemates, optically pure diastereoisomers, mixtures of diastereoisomers, diastereoisomeric racemates or mixtures of diastereoisomeric racemates.

According to the Cahn-Ingold-Prelog Convention the asymmetric carbon atom can be of the “R” or “S” configuration.

Also an embodiment of the present invention provides compounds according to formula I as described herein and pharmaceutically acceptable salts or esters thereof, in particular compounds according to formula I as described herein and pharmaceutically acceptable salts thereof, more particularly compounds according to formula I as described herein.

An embodiment of the present invention provides compounds according to formula I as described herein, wherein R¹ is H or alkoxy.

An embodiment of the present invention provides compounds according to formula I as described herein, wherein R² is 1,1,2,2-tetradeuterio-2-fluoro-ethoxy, or 1,1-dideuterio-2,2-difluoro-ethyl.

An embodiment of the present invention provides compounds according to formula I as described herein, wherein R³ is alkoxy.

An embodiment of the present invention provides compounds according to formula I as described herein, wherein R⁴ is halo or alkyl.

An embodiment of the present invention provides compounds according to formula I as described herein, wherein R⁴ is halo.

An embodiment of the present invention provides compounds according to formula I as described herein, wherein R⁵ is H or halo.

An embodiment of the present invention provides compounds according to formula I as described herein, wherein
R¹ is H or alkoxy:
R² is 1,1,2,2-tetradeuterio-2-fluoro-ethoxy, 1,1-dideuterio-2-fluoro-ethoxy, or 1,1-dideuterio-2,2-difluoro-ethyl;
R³ is alkoxy;
X₁ is CR⁵ or N;
R⁴ is H, halo;
R⁵ is H or halo;
and pharmaceutically acceptable salts.

An embodiment of the present invention provides compounds according to formula I as described herein, wherein
R¹ is H, alkoxy;
R² is 1,1,2,2-tetradeuterio-2-fluoro-ethoxy, or 1,1-dideuterio-2,2-difluoro-ethyl;
R³ is alkoxy;
X₁ is CR⁵ or N;
R⁴ is H, halo;
R⁵ is H or halo;
and pharmaceutically acceptable salts.

Particular examples of compounds of formula I as described herein are selected from
6-chloro-7-fluoro-N-[4-methoxy-5-(1,1,2,2-tetradeuterio-2-fluoro-ethoxy)pyrimidin-2-yl]-1H-indole-3-sulfonamide;
6-bromo-N-[4,6-dimethoxy-5-(1,1,2,2-tetradeuterio-2-fluoro-ethoxy)pyrimidin-2-yl]-1H-pyrrolo[2,3-b]pyridine-3-sulfonamide;
6-chloro-N-[4,6-dimethoxy-5-(1,1,2,2-tetradeuterio-2-fluoro-ethoxy) pyrimidin-2-yl]-1H-pyrrolo[2,3-b]pyridine-3-sulfonamide;
N-[5-(1,1-dideuterio-2,2-difluoro-ethyl)-4-methoxy-pyrimidin-2-yl]-6-fluoro-1H-indole-3-sulfonamide;
and pharmaceutically acceptable salts thereof.

Other particular examples of compounds of formula I as described herein are selected from
6-chloro-N-[5-(1,1-dideuterio-2,2-difluoro-ethyl)-4-methoxy-pyrimidin-2-yl]-7-fluoro 1H-indole-3-sulfonamide;
6-chloro-N-[5-(1,1-dideuterio-2-fluoro-ethoxy)-4-methoxy-pyrimidin-2-yl]-7-fluoro-1H-indole-3-sulfonamide;
6-chloro-N-[5-(1, 1-dideuterio-2,2-difluoro-ethyl)-4-methoxy-pyrimidin-2-yl]-1H-indole-3-sulfonamide;
6-bromo-N-[5-(1,1-dideuterio-2,2-difluoro-ethyl)-4-methoxy-pyrimidin-2-yl]-7-fluoro-1H-indole-3-sulfonamide;
6-bromo-N-[5-(1,1-dideuterio-2,2-difluoro-ethyl)-4,6-dimethoxy-pyrimidin-2-yl]-1H-pyrrolo [2,3-b] pyridine-3-sulfonamide;
6-bromo-N-[5-(1,1-dideuterio-2,2-difluoro-ethoxy)-4,6-dimethoxy-pyrimidin-2-yl]-1H-pyrrolo [2,3-b] pyridine-3-sulfonamide;
6-chloro-N-[5-(1, 1-dideuterio-2,2-difluoro-ethyl)-4,6-dimethoxy-pyrimidin-2-yl]-1H-pyrrolo [2,3-b] pyridine-3-sulfonamide;
N-[5-(1,1-dideuterio-2,2-difluoro-ethyl)-4,6-dimethoxy-pyrimidin-2-yl]-6-methyl-1H-pyrrolo [2,3-b] pyridine-3-sulfonamide;
and pharmaceutically acceptable salts thereof.

Processes for the manufacture of compounds of formula I as described herein are an object of the invention.

The present compounds of formula I and their pharmaceutically acceptable salts can be prepared by methods known in the art, for example, by processes described below, which process comprises

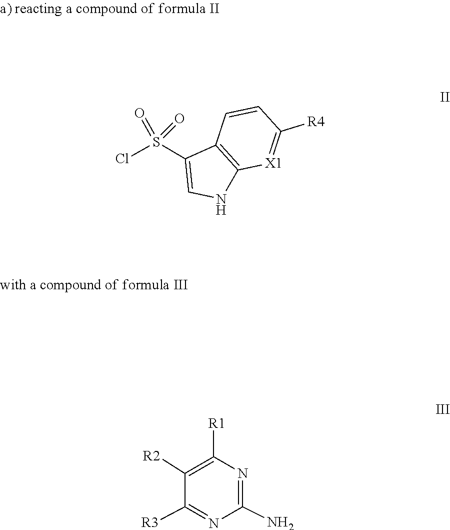

in the presence of a base selected from N,N-diisopropylethylamine, pyridine, potassium phosphate or sodium hydride to provide a compound of formula I

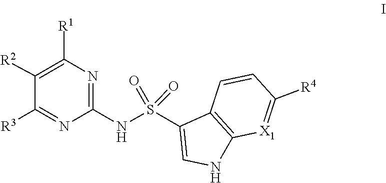

wherein the substituents R^{1,} R^{2,} R^{3,} R⁴ and X₁ are as defined above,

### General Synthetic Schemes

The compounds of formula I may be prepared in accordance with the process variant described above and with the following scheme 1. The starting materials are commercially available or may be prepared in accordance with known methods.

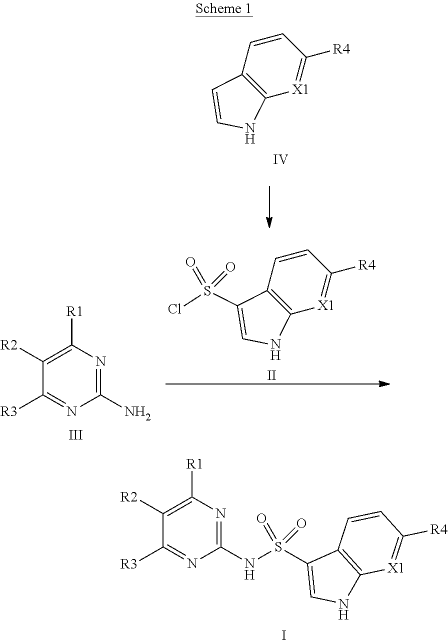

Compounds of general formula I can be prepared by reacting sulfonylchloride II with 2-amino-pyrimidine III in the presence of a base like N,N-diisopropylethylamine, pyridine, potassium phosphate or sodium hydride. II can be prepared from intermediate IV in the presence of chlorosulfonylating agent like chlorosulfonic acid, or in the presence of sulfonylating agent like sulfur trioxide N,N-dimethylformamide complex, followed by chlorination of the intermediate sulfonic acid with a chlorinating agent like thionylchloride.
2-Amino-pyrimidines of formula III may be prepared in accordance with known methods or may be prepared in accordance with the process variant described in the following scheme 2-3. The starting materials are commercially available or may be prepared in accordance with known methods.

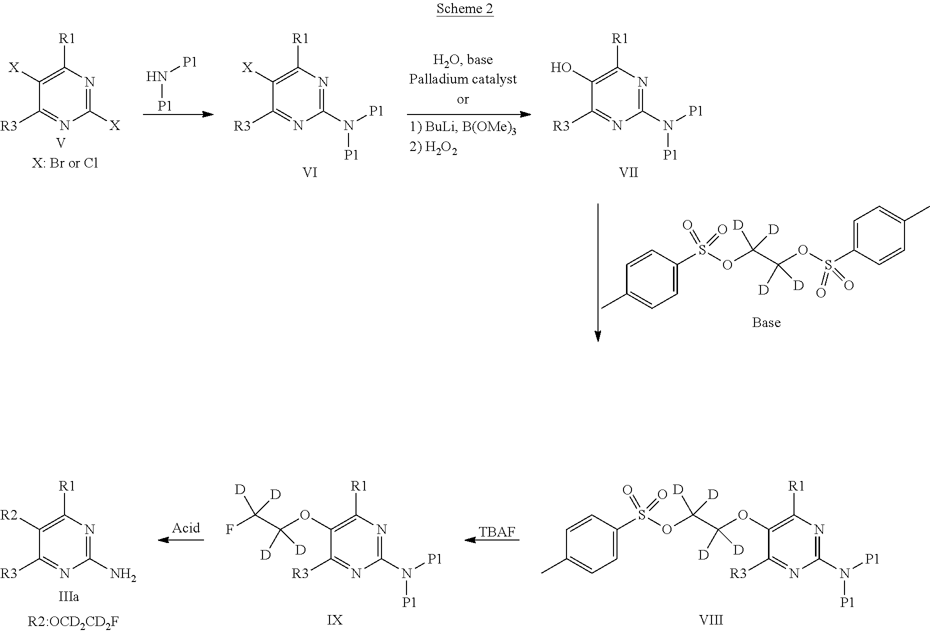

2-Amino-pyrimidine of formula IlIa wherein R² is a 1,1,2,2-tetradeuterio-2-fluoro-ethoxy group can be prepared by deprotection of intermediate IX in the presence of an acid like trifluoro acetic acid wherein PI is a protective group like p-methoxy-benzyl, 3-4-dimethoxybenzyl or a Boc group. IX can be obtained by reaction of a nucleophilic fluorine containing reagent such as TBAF with tosylate VIII. In turn VIII can be prepared by alkylation of alcohol VII in the presence of a base like cesium-or potassium-carbonate and an alkylating agent such as [1,1,2,2-tetradeuterio-2-(p-tolylsulfonyloxy)ethyl] 4-methylbenzenesulfonate. Alcohol VII can be prepared from dihalogenated starting material V, by reacting V with a protected amine to provide monohalogenated intermediate VI which is hydroxylated under Pd-catalyzed reaction condition or is first transformed into a boronic ester which is then oxidized in the presence of an oxidant like hydrogen peroxide.

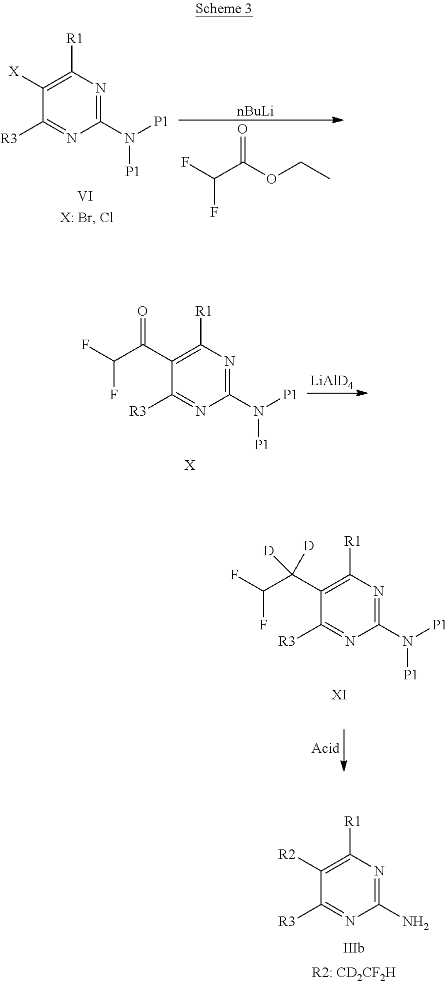

2-Amino-pyrimidine of formula IIIb wherein R² is a 1,1-dideuterio-2,2-difluoro-ethyl group can be prepared by deprotection of intermediate XI in the presence of an acid like trifluoro acetic acid wherein P1 is a protective group like p-methoxy-benzyl, 3-4-dimethoxybenzyl or a Boc group. XI can be obtained by reduction of ketone X in the a presence of a deuterated reducing agent such as lithium aluminium deuteride. X can be prepared after lithium/halogen exchange of VI and reaction with ethyl difluoroacetate.

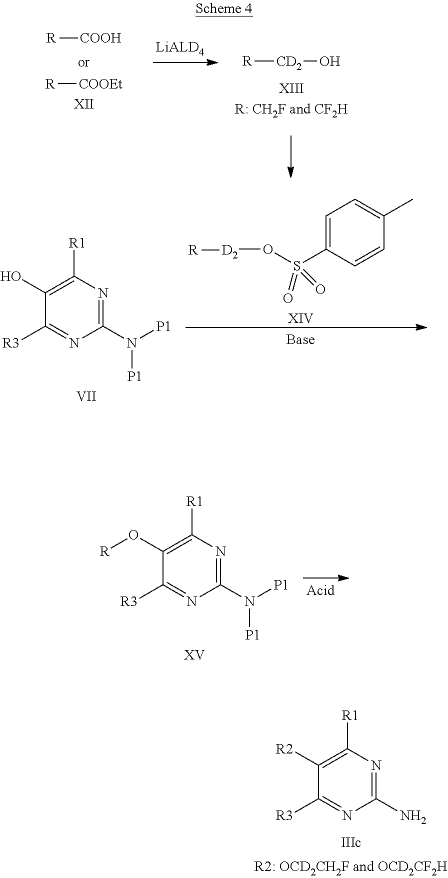

2-Amino-pyrimidine of formula IIIc wherein R² is a 1,1-dideuterio-2-fluoro-ethoxy or 1,1-dideuterio-2,2-difluoro-ethoxy group can be prepared by deprotection of intermediate XV in the presence of an acid like trifluoro acetic acid wherein P1 is a protective group like p-methoxy-benzyl, 3-4-dimethoxybenzyl or a Boc group. XV can be obtained by reaction of hydroxyprimidine compound VII with tosylate XIV in presence of a base like cesium- or potassium carbonate. In turn XIV can be prepared by reduction of a suitable fluorine containing carboxylic acid or carboxylic ester XII with lithium aluminium deuteride to give alcohol XIII, followed by reaction with p-toluene sulfonyl chloride XIV in the presence of a base.

Another embodiment of the invention provides a pharmaceutical composition or medicament containing a compound of the invention and a therapeutically inert carrier, diluent or excipient, as well as a method of using the compounds of the invention to prepare such composition and medicament. In one example, the compound of formula I may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula I is formulated in an acetate buffer, at pH 5. In another embodiment, the compound of formula I is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

The compounds of formula I and their pharmaceutically acceptable salts can be processed with pharmaceutically inert, inorganic or organic adjuvants for the production of tablets, coated tablets, dragées, hard gelatin capsules, injection solutions or topical formulations Lactose, corn starch or derivatives thereof, talc, stearic acid or its salts etc. can be used, for example, as such adjuvants for tablets, dragées and hard gelatin capsules.

Suitable adjuvants for soft gelatin capsules, are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Suitable adjuvants for topical ocular formulations are, for example, cyclodextrins, mannitol or many other carriers and excipients known in the art.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should it be appropriate. In the case of topical administration, the formulation can contain 0.001% to 15% by weight of medicament and the required dose, which can be between 0.1 and 25 mg in can be administered either by single dose per day or per week, or by multiple doses (2 to 4) per day, or by multiple doses per week It will, however, be clear that the upper or lower limit given herein can be exceeded when this is shown to be indicated.

The invention also relates in particular to:
A compound of formula I for use as therapeutically active substance.
A compound of formula I for use in the treatment of a disease modulated by GPR17.

Likewise an object of the present invention is a pharmaceutical composition comprising a compound according to formula I as described herein and a therapeutically inert carrier.

The use of a compound of formula I for the treatment or prophylaxis of conditions resulting from direct damage to myelin sheaths (including but not limited central pontine and extra-pontine myelinolysis, carbon monoxide poisoning, nutritional deficiency, and virus-induced demyelination), demyelinating disorders (including but not limited to multiple sclerosis, acute and multiphasic disseminated encephalomyelitis, neuromyelitis optica spectrum disorders. and leukodystrophies), CNS disorders associated with myelin loss (including but not limited to Alzheimer's disease, schizophrenia, Parkinson's disease, Huntington's disease, Amyotrophic lateral sclerosis, and Ischemia due to stroke), and Inflammation in the CNS for instance following encephalitis, primary angiitis, meningitis and obesity.

An embodiment of the present invention is the use of a compound of formula I for the treatment or prophylaxis of multiple sclerosis, Alzheimer's disease, Parkinson's disease, or Huntington's disease.

A particular embodiment of the invention is the use of a compound of formula I for the treatment or prophylaxis of multiple sclerosis.

The use of a compound of formula I for the preparation of a medicament for the treatment or prophylaxis of conditions resulting from direct damage to myelin sheaths (including but not limited central pontine and extra-pontine myelinolysis, carbon monoxide poisoning, nutritional deficiency, and virus-induced demyelination), demyelinating disorders (including but not limited to multiple sclerosis, acute and multiphasic disseminated encephalomyelitis, neuromyelitis optica spectrum disorders, and leukodystrophies), CNS disorders associated with myelin loss (including but not limited to Alzheimer's disease, schizophrenia, Parkinson's disease, Huntington's disease, Amyotrophic lateral sclerosis, and Ischemia due to stroke), and Inflammation in the CNS for instance following encephalitis, primary angiitis, meningitis and obesity.

An embodiment of the present invention is the use of a compound of formula I for the preparation of a medicament for the treatment or prophylaxis of multiple sclerosis, Alzheimer's disease, Parkinson's disease, or Huntington's disease.

A particular embodiment of the invention is the use of a compound of formula I for the preparation of a medicament for the treatment or prophylaxis of multiple sclerosis.

A compound according to formula I for use in the treatment or prophylaxis of conditions resulting from direct damage to myelin sheaths (including but not limited central pontine and extra-pontine myelinolysis, carbon monoxide poisoning, nutritional deficiency, and virus-induced demyelination), demyelinating disorders (including but not limited to multiple sclerosis, acute and multiphasic disseminated encephalomyelitis, neuromyelitis optica spectrum disorders, and leukodystrophies), CNS disorders associated with myelin loss (including but not limited to Alzheimer's disease, schizophrenia, Parkinson's disease, Huntington's disease, Amyotrophic lateral sclerosis, and Ischemia due to stroke), and Inflammation in the CNS for instance following encephalitis, primary angiitis, meningitis and obesity.

An embodiment of the present invention is a compound of formula I for use in the treatment or prophylaxis of multiple sclerosis, Alzheimer's disease, Parkinson's disease, or Huntington's disease.

A particular embodiment of the invention is a compound according to formula I for use in the treatment or prophylaxis of multiple sclerosis.

A method for the treatment or propylaxis of conditions resulting from direct damage to myelin sheaths (including but not limited central pontine and extra-pontine myelinolysis, carbon monoxide poisoning, nutritional deficiency, and virus-induced demyelination), demyelinating disorders (including but not limited to multiple sclerosis, acute and multiphasic disseminated encephalomyelitis, neuromyelitis optica spectrum disorders, and leukodystrophies), CNS disorders associated with myelin loss (including but not limited to Alzheimer's disease, schizophrenia, Parkinson's disease, Huntington's disease, Amyotrophic lateral sclerosis, and Ischemia due to stroke), and Inflammation in the CNS for instance following encephalitis, primary angiitis, meningitis and obesity, which method comprises administering an effective amount of a compound of formula I to a patient in need thereof.

An embodiment of the present invention is a method for the treatment or prophylaxis of multiple sclerosis, Alzheimer's disease, Parkinson's disease, or Huntington's disease, which method comprises administering an effective amount of a compound of formula I to a patient in need thereof.

A particular embodiment of the invention is a method for the treatment or prophylaxis of multiple sclerosis, which method comprises administering an effective amount of a compound of formula I to a patient in need thereof.

Also an embodiment of the present invention provides compounds of formula I as described herein, when manufactured according to any one of the described processes.

### Assay Procedures

### GPR17 CAMP Assay Protocol:

CHO-KI cells stably expressing vector containing untagged human GPR17 short isoform (Roche) were cultured at 37° C./5% CO2 in DMEM (Dulbecco's Modified Eagle Medium): F-12 (1:1) supplemented with 10% foetal bovine serum and 400 g/ml Geneticin.

Changes in intracellular cyclic adenosine monophosphate (cAMP) levels were quantified using the Nano-TRF Detection Assay kit (Roche Diagnostics, Cat. No. 05214386001). This assay allows for direct cAMP quantification in a homogeneous solution. cAMP is detected based on time-resolved fluorescence energy transfer (TR-FRET) and competitive binding of ruthenylated cAMP and endogenous cAMP to an anti-cAMP monoclonal antibody labeled with AlexaFluor-700. The Ruthenium complex serves as the FRET donor and transfers energy to AlexaFluor-700. The FRET signal is inversely proportional to the cAMP concentration.

CHO-GPR17S cells were detached with Accutase and resuspended in assay buffer consisting of Hank's Balanced Salt Solution (HBSS), 10 mM HEPES (4-(2-hydroxyethyl) piperazine-1-ethanesulfonic acid solution) and 0.1% bovine serum albumin (pH 7.4). The cells were seeded in black 384-well plates (Corning) at a density of 10,000 cells/20 μl assay buffer until the addition of compounds.

Test antagonist compounds were serially diluted in dimethyl sulfoxide (DMSO) and spotted in 384-well plates. The compounds were then diluted in HBSS buffer supplemented with an EC80 concentration of MDL29,951 (3-(2-Carboxy-4,6-dichloroindol-3-yl) propionic acid) (GPR17 agonist) plus 3-Isobutyl-1-methylxanthine (IBMX) (0.5 mM final concentration) and added to the cells at room temperature. Forskolin (15 μM final concentration) was added 5 minutes after the test compounds and the cells were incubated at room temperature for 30 minutes. The assay was stopped by adding cAMP detection mix (containing detergents for cell lysis) for 90 minutes at room temperature.

Cellular cAMP was measured using a Paradigm reader (Molecular Devices). The raw data was used to calculate the FRET signal based on the assay's P-factor as per cAMP kit instructions. The data was normalized to the maximal activity of a reference antagonist and dose response curves were fitted to the percent activity of the test compounds using a sigmoidal dose response model (Genedata Screener).

Results in the hGPR17 CAMP assay are provided for compounds of formula I in Table 1

TABLE 1

| Example | hGPR17 CAMP |
|---|---|
| number | IC50 [μM] |
| 1 | 0.085 |
| 2 | 0.042 |
| 3 | 0.063 |
| 4 | 0.094 |
| 5 | 0.107 |
| 6 | 0.105 |
| 7 | 0.055 |
| 8 | 0.137 |
| 9 | 0.023 |
| 10 | 0.081 |
| 11 | 0.037 |
| 12 | 0.057 |

The invention will now be illustrated by the following examples which have no limiting character.

In case the preparative examples are obtained as a mixture of enantiomers, the pure enantiomers can be obtained by methods described herein or by methods known to those skilled in the art, such as e.g. chiral chromatography or crystallization.

### EXAMPLES

All examples and intermediates were prepared under nitrogen atmosphere if not specified otherwise.

### Intermediates A

The intermediates A1-A4 are known and have been prepared following procedures described in the indicated patent applications:

| | | | Patent |
|---|---|---|---|
| Intermediate | | | application |
| number | Structure | Name (CAS number) | number |
| A1 | 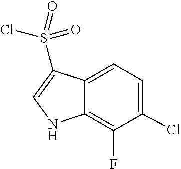 [SMILES: O=S(=O)(Cl)c1c[nH]c2c(F)c(Cl)ccc12] | 6-chloro-7-fluoro-1H-indole- 3-sulfonyl chloride (2231234-36-7) | WO2018122233 |
| A2 | 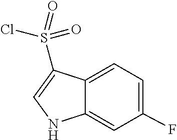 [SMILES: O=S(=O)(Cl)c1c[nH]c2cc(F)ccc12] | 6-fluoro-1H-indole-3-sulfonyl chloride (1216026-07-1) | WO2018122232 |
| A3 | 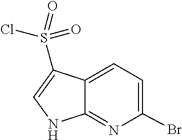 [SMILES: O=S(=O)(Cl)c1c[nH]c2nc(Br)ccc12] | 6-bromo-1H-pyrrolo[2,3- b]pyridine-3-sulfonyl chloride (2231234-27-6) | WO2018122232 |
| A4 | 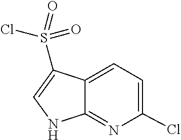 [SMILES: O=S(=O)(Cl)c1c[nH]c2nc(Cl)ccc12] | 6-chloro-1H-pyrrolo[2,3- b]pyridine-3-sulfonyl chloride (2231234-21-0) | WO2018122233 |
| A5 | 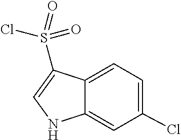 [SMILES: O=S(=O)(Cl)c1c[nH]c2cc(Cl)ccc12] | 6-chloro-1H-indole-3-sulfonyl chloride (1216060-79-5) | WO2019243303 |
| A6 | 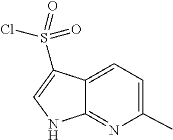 | 6-methyl-1H-pyrrolo[2,3- b]pyridine-3-sulfonyl chloride (2827057-38-3) | WO2022180136 |
| A7 | 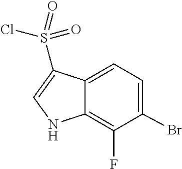 [SMILES: O=S(=O)(Cl)c1c[nH]c2c(F)c(Br)ccc12] | 6-bromo-7-fluoro-1H-indole- 3-sulfonyl chloride (2827057-05-4) | WO2022180136 |

### Intermediates B

### Intermediate B1: 4-methoxy-5-(1,1,2,2-tetradeuterio-2-fluoro-ethoxy)pyrimidin-2-amine

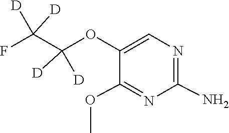 [SMILES: [2H]C([2H])(F)C([2H])([2H])Oc1cnc(N)nc1OC]

Step 1: 5-bromo-N,N-bis[(2,4-dimethoxyphenyl)methyl]-4-methoxy-pyrimidin-2-amine

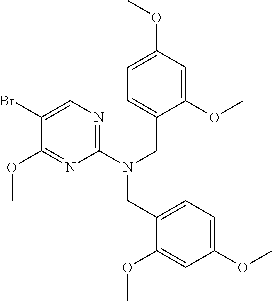 [SMILES: COc1ccc(CN(Cc2ccc(OC)cc2OC)c2ncc(Br)c(OC)n2)c(OC)c1]

To a solution of 5-bromo-2-chloro-4-methoxy-pyrimidine (50.0 g, 223.75 mmol) in DMF (400 mL) was added 1-(2,4-dimethoxyphenyl)-N- [(2,4-dimethoxyphenyl) methyl]methanamine (78.12 g, 246.13 mmol) and potassium carbonate (61.85 g, 447.51 mmol), then the mixture was stirred at 80° C. for 16 h. The reaction mixture was diluted with water (500 mL) and extracted with ethyl acetate (500 mL×2). The combined organic layers were washed with brine (500 mL×2), dried over sodiumsulfate, filtered and concentrated under reduced pressure. The residue was purified by flash chromatography over silica gel using a gradient ethylacetate/ether 0-100% to provide the title compound as a white solid (77g, 65% yield). MS (ESI): m/z=504.1 [M+H]⁺

Step 2: 2-[bis[(2,4-dimethoxyphenyl)methyl]amino]-4-methoxy-pyrimidin-5-ol

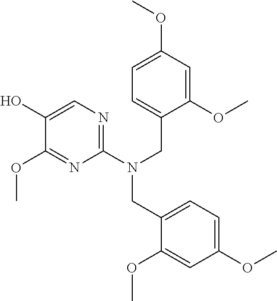 [SMILES: COc1ccc(CN(Cc2ccc(OC)cc2OC)c2ncc(O)c(OC)n2)c(OC)c1]

A mixture of 5-bromo-N,N-bis[(2,4-dimethoxyphenyl) methyl]-4-methoxy-pyrimidin-2-amine (10.0 g, 19.83 mmol) and sodium tert-butoxide (5716.24 mg, 59.48 mmol) in 1,4-Dioxane (10 mL) and water (1 mL) was added TBUBRETTPHOS PD G3 (1000.0 mg, 1.17 mmol). The reaction mixture was degassed with nitrogen for 1 minute and heated to 120° C., stirred at this temperature for 16 h under nitrogen. The reaction mixture was filtered and concentrated under reduced pressure. The residue was purified by flash chromatography over silica gel using a gradient ethylacetate/ether 0-50% to provide the title compound as a black solid (10 g, 95% yield). MS (ESI): m/z=442.1 [M+H]+

Step 3: [2-[2-[bis[(2,4-dimethoxyphenyl)methyl]amino]-4-methoxy-pyrimidin-5-yl]oxy-1,1,2,2-tetradeuterio-ethyl]4-methylbenzenesulfonate

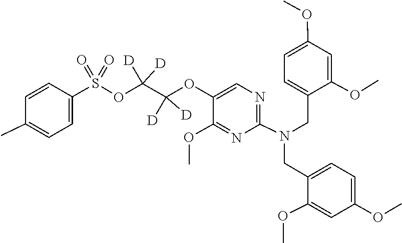

To a solution of 2-[bis[(2,4-dimethoxyphenyl)methyl]amino]-4-methoxy-pyrimidin-5-ol (2.0 g, 4.53 mmol) and potassiumcarbonate (1.25 g, 9.06 mmol) in DMF (15 mL) was added [1,1,2,2-tetradeuterio-2-(p-tolylsulfonyloxy)ethyl]4-methylbenzenesulfonate (2.04 g, 5.44 mmol, CAS: 164936-35-0) the reaction was stirred vigorously at 70° C. for 2 h. The reaction mixture was transferred to a separatory funnel, the organic layer was separated, and the aqueous layer was extracted with ethylacetate (3×100 mL). The combined organic phases were dried over sodiumsulfate and filtered, concentrated in vacuo to provide the title compound as a yellow oil (2.9 g, 99% yield) which was engaged directly in the next step without further purification.

Step 4: N,N-bis[(2,4-dimethoxyphenyl) methyl]-4-methoxy-5-(1,1,2,2-tetradeuterio-2-fluoro-ethoxy) pyrimidin-2-amine

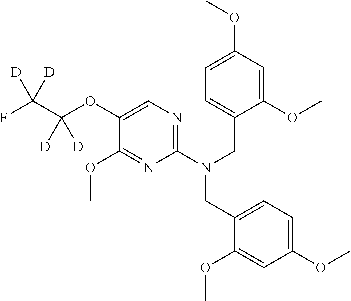 [SMILES: [2H]C([2H])(F)C([2H])([2H])Oc1cnc(N(Cc2ccc(OC)cc2OC)Cc2ccc(OC)cc2OC)nc1OC]

To a solution of [2-[2-[bis[(2,4-dimethoxyphenyl) methyl]amino]-4-methoxy-pyrimidin-5-yl]oxy-1,1,2,2-tetradeuterio-ethyl]4-methylbenzenesulfonate (2.9 g, 4.5 mmol) in acetonitrile (20 mL) was added TBAF (13.51 mL, 13.51 mmol) at 25° C. The mixture was heated to 85° C. for 1 h. The reaction mixture was transferred to a separatory funnel, the organic layer was separated, and the aqueous layer was extracted with ethylacetate (3×100 mL). The combined organic phase was dried over sodiumsulfate and filtered, concertrated in vacuo. The residue was purified by flash chromatography over silica gel using a gradient ethylacetate/ether 0-30% to provide the title compound as a light yellow oil (1.3g, 56% yield). MS (ESI): m/z=492.2 [M+H]+

Step 5: 4-methoxy-5-(1,1,2,2-tetradeuterio-2-fluoro-ethoxy)pyrimidin-2-amine

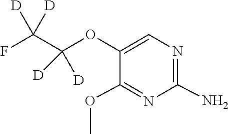 [SMILES: [2H]C([2H])(F)C([2H])([2H])Oc1cnc(N)nc1OC]

To a solution of N,N-bis[(2,4-dimethoxyphenyl) methyl]-4-methoxy-5-(1,1,2,2-tetradeuterio-2-fluoro-ethoxy)pyrimidin-2-amine (11.0 g, 22.38 mmol) in dichloromethane (35 mL) was added TFA (35.0 mL) at 0° C. The mixture was stirred at 25° C. for 2 h. quenched with a saturated aqueous solution of sodiumbicarbonate (600 mL) and extracted with ethylacetate (3×200 mL). The organic layers were dried over sodium sulfate, filtered and concentrated in vacuo. The residue was mixed with petroleum ether (30 mL) stirred at 25° C. for 30 min and filtered to provide the title compound as a white solid (2.3g, 53% yield). MS (ESI): m/z=192.1 [M+H]+

### Intermediate B2:4,6-dimethoxy-5-(1,1,2,2-tetradeuterio-2-fluoro-ethoxy) pyrimidin-2-amine

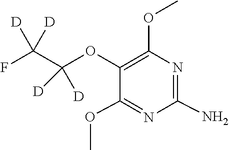 [SMILES: [2H]C([2H])(F)C([2H])([2H])Oc1c(OC)nc(N)nc1OC]

Step 1: N,N-bis[(2,4-dimethoxyphenyl)methyl]-4,6-dimethoxy-pyrimidin-2-amine

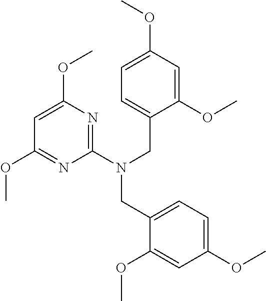 [SMILES: COc1ccc(CN(Cc2ccc(OC)cc2OC)c2nc(OC)cc(OC)n2)c(OC)c1]

To a solution of 2-chloro-4,6-dimethoxypyrimidine (20.0 g, 114.56 mmol) and 1-(2,4-dimethoxyphenyl)-N-[(2,4-dimethoxyphenyl) methyl]methanamine (43.63 g, 137.47 mmol) in NMP (400 mL) was added cesiumcarbonate (74.69 g, 229.12 mmol). The mixture was stirred at 120° C. for 16 h. The reaction mixture was diluted with water (500 mL) and extracted with ethyl acetate (500 mL×2). The combined organic layers were washed with brine (200 mL×2), dried over sodiumsulfate, filtered and concentrated in vacuo. The residu was triturated in ethyl acetate.

The obtained solid was filtered and dried to provide 25 g of the title compound. The filtrate was concentrated in vacuo. The residue was purified by flash chromatography over silica gel using a gradient of ethylacetate/ether 0-100% to provide a white solid. This solid was combined with the solid obtained above by filtration to provide the title compound as a white solid (43g, 75% yield). MS (ESI): m/z=456.2 [M+H]+

Step 2: 5-bromo-N,N-bis[(2,4-dimethoxyphenyl)methyl]-4,6-dimethoxy-pyrimidin-2-amine

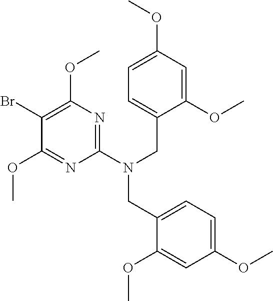 [SMILES: COc1ccc(CN(Cc2ccc(OC)cc2OC)c2nc(OC)c(Br)c(OC)n2)c(OC)c1]

To a solution of N,N-bis[(2,4-dimethoxyphenyl)methyl]-4,6-dimethoxy-pyrimidin-2-amine (32.0 g, 70.25 mmol) in MeCN (320 mL) was added N-bromosuccinimide (11.25 g, 63.23 mmol). The mixture was stirred at 20° C. for 3 h. After completion of the reaction, the mixture was quenched by sodium sulfite (500 mL) aqueous solution and stirred at 20° C. for 0.5 h. The mixture was extracted with dichloromethane (500 mL×2). The combined organic layers were washed with brine (500 mL×2), dried over sodium sulfate, filtered and concentrated in vacuo. The gum was triturated in ethyl acetate, the formed precipitate was filtered to provide the title compound as a white solid (28g, 62% yield). MS (ESI): m/z=534.2 [M+H]+

Step 3: 2-[bis[(2,4-dimethoxyphenyl)methyl]amino]-4,6-dimethoxy-pyrimidin-5-ol

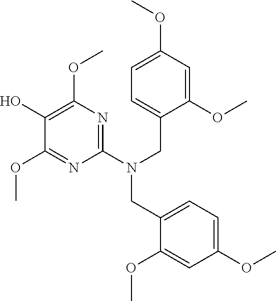 [SMILES: COc1ccc(CN(Cc2ccc(OC)cc2OC)c2nc(OC)c(O)c(OC)n2)c(OC)c1]

To a solution of 5-bromo-N,N-bis[(2,4-dimethoxyphenyl) methyl]-4,6-dimethoxy-pyrimidin-2-amine (33.0 g, 61.75 mmol) in THF (650 mL) was added n-BuLi (32.11 mL, 80.28 mmol) at −70° C. The mixture was stirred at −70° C. for 0.5 h. Then Trimethyl borate (13.81 mL, 123.5 mmol) was added at −70° C. and the mixture was stirred at −70° C. for 2 h. The mixture was warmed to 0° C. and acetic acid (7.84 mL, 135.85 mmol), hydrogen peroxide (13.87 mL, 135.85 mmol) were added at 0° C. The mixture was stirred at 20° C. for 2 h. After completion of the reaction, the mixture was quenched by sodium sulfite (500 mL) aqueous solution, stirred at 20° C. for 0.5 h and extracted with ethyl acetate (20 mL×2). The combined organic layers were washed with brine (20 mL×2), dried over sodium sulfate, filtered and concentrated in vacuo. The residue was purified by flash chromatography over silica gel using a gradient ethylacetate/ether 0-50% to provide the title compound as a yellow oil (23.8g, 76% yield). MS (ESI): m/z=472.3 [M+H]+

Step 4: [2-[2-[bis[(2,4-dimethoxyphenyl)methyl]amino]-4.6-dimethoxy-pyrimidin-5-yl]oxy-1,1,2,2-tetradeuterio-ethyl]4-methylbenzenesulfonate

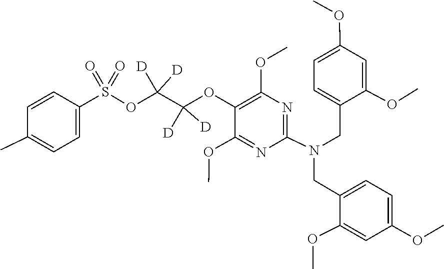

The title compound was prepared in analogy to intermediate B1 step 3 from 2-[bis[(2,4-dimethoxyphenyl) methyl]amino]-4,6-dimethoxy-pyrimidin-5-ol as a white solid. MS (ESI): m/z=674.4 [M+H]+

Step 5: N,N-bis[(2,4-dimethoxyphenyl)methyl]-4.6-dimethoxy-5-(1,1,2,2-tetradeuterio-2-fluoro-ethoxy) pyrimidin-2-amine

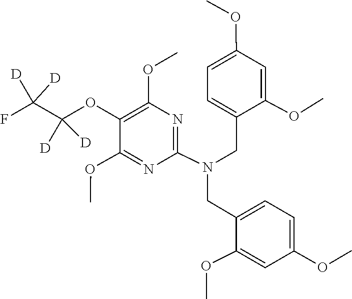 [SMILES: [2H]C([2H])(F)C([2H])([2H])Oc1c(OC)nc(N(Cc2ccc(OC)cc2OC)Cc2ccc(OC)cc2OC)nc1OC]

The title compound was prepared in analogy to intermediate B1 step 4 from [2-[2-[bis[(2,4-dimethoxyphenyl)methyl]amino]-4,6-dimethoxy-pyrimidin-5-yl]oxy-1,1,2,2-tetradeuterio-ethyl]4-methylbenzenesulfonate as a white solid. MS (ESI): m/z=522.4 [M+H]+

Step 6: 4,6-dimethoxy-5-(1,1,2,2-tetradeuterio-2-fluoro-ethoxy)pyrimidin-2-amine

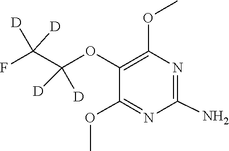 [SMILES: [2H]C([2H])(F)C([2H])([2H])Oc1c(OC)nc(N)nc1OC]

The title compound was prepared in analogy to intermediate B1 step 5 from N,N-bis[(2,4-dimethoxyphenyl) methyl]-4,6-dimethoxy-5-(1,1,2,2-tetradeuterio-2-fluoro-ethoxy) pyrimidin-2-amine as a white solid. MS (ESI): m/z=222.2 [M+H]+

### Intermediate B3:5-(1,1-dideuterio-2,2-difluoro-ethyl)-4-methoxy-pyrimidin-2-amine

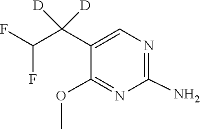

Step 1: 1-[2-[bis[(2,4-dimethoxyphenyl)methyl]amino]-4-methoxy-pyrimidin-5-yl]-2,2-difluoro-ethanone

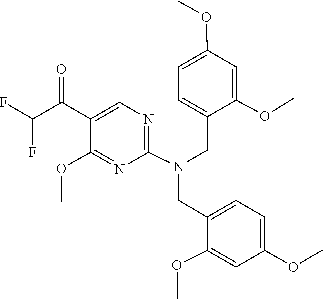 [SMILES: COc1ccc(CN(Cc2ccc(OC)cc2OC)c2ncc(C(=O)C(F)F)c(OC)n2)c(OC)c1]

To a solution of 5-bromo-N,N-bis[(2,4-dimethoxyphenyl)methyl]-6-methoxy-pyridin-2-amine (2000.0 mg, 3.97 mmol, intermediate B1 step 1) in THF (3 mL) was added n-BuLi (1.91 mL, 4.77 mmol) in portions at −70° C., and the solution was stirred at −70° C. for 0.5 hr. Then ethyl difluoroacetate (1.31 mL, 11.92 mmol) was added in protions at −70° C. The solution was stirred at 20° C. for 2 hr. The reaction mixture was poured into a saturated ammonium chloride solution (250 mL) and extracted with ethylacetate (150ml×3). The organic layer was washed with brine, dried over sodium sulfate and concentrated in vacuo. The residue was purified by flash chromatography over silica gel using a gradient ethylacetate/ether 0-30% to provide the title compound as a yellow oil (0.8 g, 33% yield). MS (ESI): m/z=504.1 [M+H]+

Step 2: 5-(1,1-dideuterio-2,2-difluoro-ethyl)-N,N-bis[(2,4-dimethoxyphenyl) methyl]-4-methoxy-pyrimidin-2-amine

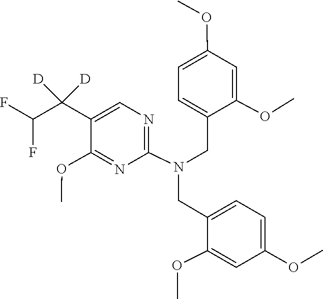 [SMILES: [2H]C([2H])(c1cnc(N(Cc2ccc(OC)cc2OC)Cc2ccc(OC)cc2OC)nc1OC)C(F)F]

To a solution of LiAlD4 (2.0 g, 47.67 mmol) and AlCl₃ (6.99 g, 52.43 mmol) in diethyl ether (200 mL) stirred for 15 min under a nitrogen atmosphere, was added a solution of 1-[2-[bis[(2,4-dimethoxyphenyl) methyl]amino]-4-methoxy-pyrimidin-5-yl]-2,2-difluoro-ethanone (24.0 g, 47.67 mmol) in diethyl ether (50 mL). The mixture was stirred at 20° C. for 2 h, quenched with D₂O (100 mL) followed by 6N sulfuric acid (100 mL) and then diluted with water (150 mL). The aqueous layer was extracted with ethylacetate (3×300 mL), the combined organic layers were washed with water, and with a 10% sodium bicarbonate aqueous solution. The combined organic phases were dried over sodium sulfate, filtered, and concentrated in vacuo. The residue was purified by flash chromatography over silica gel using a gradient ethylacetate/ether 0-30% to provide the title compound as a yellow oil (15 g, 64% yield). MS (ESI): m/z=492.2 [M+H]+

Step 3: 5-(1,1-dideuterio-2,2-difluoro-ethyl)-4-methoxy-pyrimidin-2-amine

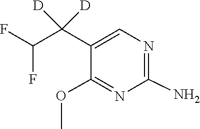

The title compound was prepared in analogy to intermediate B1 step 5 from 5-(1,1-dideuterio-2,2-difluoro-ethyl)-N,N-bis[(2,4-dimethoxyphenyl) methyl]-4-methoxy-pyrimidin-2-amine as a white solid. MS (ESI): m/z=192.1 [M+H]+

### Intermediate B4: 5-(1,1-dideuterio-2-fluoro-ethoxy)-4-methoxy-pyrimidin-2-amine

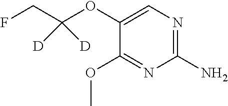

Step 1: (1,1-dideuterio-2-fluoro-ethyl)4-methylbenzenesulfonate

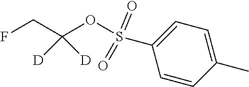 [SMILES: [2H]C([2H])(CF)OS(=O)(=O)c1ccc(C)cc1]

To a solution of ethyl fluoroacetate (500 mg, 4.71 mmol) in tetrahydrofuran (10 ml) was added lithium aluminium deuteride (198 mg, 4.71 mmol) at 0-10° C. in portions and the mixture was stirred at 20° C. for 1 h. Then water (170 mg, 9.43 mmol) was carefully added dropwise, followed by Na2SO4 (1 g). The mixture was filtered and washed with dichloromethane (10 ml). To the filtrate were added triethylamine (1.96 ml, 14.14 mmol) and p-toluenesulfonyl chloride (1.35 g, 7.07 mmol) at 0-10° C. and the mixture was stirred at 20° C. for 16 h. The reaction mixture was diluted with dichloromethane (30 ml), washed with brine, dried over Na2SO4 and concentrated under reduced pressure. The residue was purified by column chromatography (silica gel, petroleum ether/ethyl acetate=1:0 to 5:1) to give (1,1-dideuterio-2-fluoro-ethyl) 4-methylbenzenesulfonate (230 mg, 21% yield) as colorless oil.

Step 2: 5-(1,1-dideuterio-2-fluoro-ethoxy)-N,N-bis[(2,4-dimethoxyphenyl) methyl]-4-methoxy-pyrimidin-2-amine

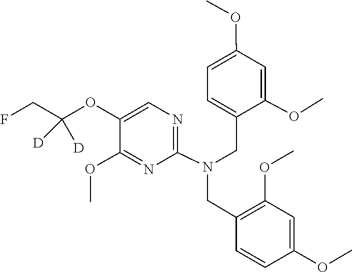 [SMILES: [2H]C([2H])(CF)Oc1cnc(N(Cc2ccc(OC)cc2OC)Cc2ccc(OC)cc2OC)nc1OC]

To a mixture of 2-[bis[(2,4-dimethoxyphenyl) methyl]amino]-4-methoxy-pyrimidin-5-ol (2.5 g, 5.66 mmol, see Intermediate B1) and cesium carbonate (3.69 g, 11.3 mmol) in dimethylformamide (50 ml) was added (1,1-dideuterio-2-fluoro-ethyl) 4-methylbenzenesulfonate (1.7 g, 7.72 mmol) and the mixture was stirred at 60° C. for 2 h. Then the mixture was diluted with water (200 ml) and extracted with ethyl acetate twice. The combined organic layers were washed with brine, dried over Na2SO4 and concentrated under reduced pressure. The residue was purified by column chromatography (silica gel, petroleum ether/ethyl acetate=1:0 to 5:1) to give 5-(1, 1-dideuterio-2-fluoro-ethoxy)-N,N-bis[(2,4-dimethoxyphenyl) methyl]-4-methoxy-pyrimidin-2-amine (1.5 g, 53% yield) as brown oil, MS (ESI) m/z=490.3 [M+H]+.

Step 3:5-(1,1-dideuterio-2-fluoro-ethoxy)-4-methoxy-pyrimidin-2-amine

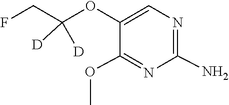

To a solution of 5-(1,1-dideuterio-2-fluoro-ethoxy)-N,N-bis[(2,4-dimethoxyphenyl)methyl]-4-methoxy-pyrimidin-2-amine (1.40 g, 2.86 mmol) in dichloromethane (10 ml) was added trifluoroacetic acid (10 ml, 135 mmol) at 0-10° C. and the mixture was stirred at 20° C. for 2 h. Then the pH of the mixture was adjusted to pH 7 with saturated aqueous NaHCO3 solution and extracted with ethyl acetate twice. The combined organic layers were washed with brine, dried over Na2SO4 and concentrated under reduced pressure. The residue was triturated with ethyl acetate (2 ml) and filtered. The filter cake was collected to give 5-(1,1-dideuterio-2-fluoro-ethoxy)-4-methoxy-pyrimidin-2-amine (467 mg, 85% yield) as a white solid, MS (ESI) m/z=190.2 [M+H]⁺.

### Intermediate B5: 5-(1,1-dideuterio-2,2-difluoro-ethyl)-4,6-dimethoxy-pyrimidin-2-amine

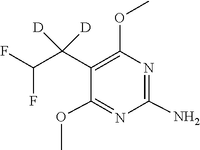 [SMILES: [2H]C([2H])(c1c(OC)nc(N)nc1OC)C(F)F]

The title compound was prepared in analogy to Intermediate B3 from 5-bromo-N,N-bis [(2,4-dimethoxyphenyl) methyl]-4,6-dimethoxy-pyrimidin-2-amine (see Intermediate B2) instead of 5-bromo-N, N-bis[(2,4-dimethoxyphenyl) methyl]-6-methoxy-pyridin-2-amine in step 1) as white solid. MS (ESI) m/z: 222.1 [M+H]⁺.

### Intermediate B6: 5-(1,1-dideuterio-2,2-difluoro-ethoxy)-4,6-dimethoxy-pyrimidin-2-amine

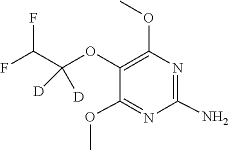 [SMILES: [2H]C([2H])(Oc1c(OC)nc(N)nc1OC)C(F)F]

Step 1:1,1-dideuterio-2,2-difluoro-ethanol

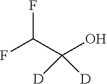

To the suspension of lithium aluminium deuteride (437 mg, 10.4 mmol) in tetrahydrofuran (20 ml) was added slowly difluoroacetic acid (1.0 g, 10.4 mmol) at 0° C. under nitrogen, then the mixture was stirred at 60° C. for 2 h under nitrogen atmosphere. After cooling to 20° C., wet Na2SO4 was added to the above mixture until bubbling ceased. Then dichloromethane (30 ml) and dry Na2SO4 was added and the mixture was stirred for 5 min, then filtered and evaporated carefully. A colorless liquid was obtained (875 mg, 99% yield) which was directly used for the next step.

Step 2: (1,1-dideuterio-2,2-difluoro-ethyl) 4-methylbenzenesulfonate

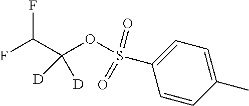 [SMILES: [2H]C([2H])(OS(=O)(=O)c1ccc(C)cc1)C(F)F]

To a solution of p-toluenesulfonyl chloride (2.38 g, 12.5 mmol) in dichloromethane (10 ml) were added triethylamine (2.16 ml, 15.6 mmol), N,N-dimethylpyridin-4-amine (64 mg, 0.52 mmol, 0.05 eq) and 1,1-dideuterio-2,2-difluoro-ethanol (875 mg, 10.4 mmol) and the mixture was stirred at 20° C. for 12 h. The reaction mixture was quenched by addition of saturated. NaHCO3 solution (80 ml) at 20° C., and then extracted with ethyl acetate (50 ml×3). The combined organic layers were washed with brine, dried over Na2SO4, filtered and concentrated under reduced pressure. The residue was purified by flash chromatography (silica gel, ethyl acetate/petroleum ether) to give (1,1-dideuterio-2,2-difluoro-ethyl) 4-methylbenzenesulfonate (1.44 g, 58% yield) as a colorless oil, MS (ESI) m/z: 238.8 [M+H]⁺.

Step 3: 5-(1,1-dideuterio-2,2-difluoro-ethoxy)-N,N-bis[(2,4-dimethoxyphenyl)methyl]-4,6-dimethoxy-pyrimidin-2-amine

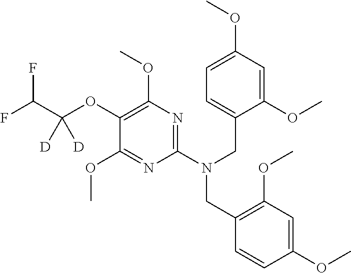 [SMILES: [2H]C([2H])(Oc1c(OC)nc(N(Cc2ccc(OC)cc2OC)Cc2ccc(OC)cc2OC)nc1OC)C(F)F]

The title compound was prepared in analogy to Intermediate B4 from 2-[bis[(2,4-dimethoxyphenyl)methyl]amino]-4,6-dimethoxy-pyrimidin-5-ol (see Intermediate B2) instead of 2-[bis[(2,4-dimethoxyphenyl)methyl]amino]-4-methoxy-pyrimidin-5-ol and (1,1-dideuterio-2,2-difluoro-ethyl) 4-methylbenzenesulfonate instead of (1,1-dideuterio-2-fluoro-ethyl) 4-methylbenzenesulfonate in step 2) as a yellow solid, MS (ESI): m/z=538.2 [M+H]⁺.

Step 4: 5-(1,1-dideuterio-2.2-difluoro-ethoxy)-4.6-dimethoxy-pyrimidin-2-amine

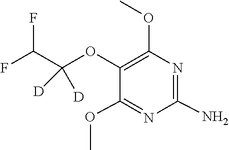 [SMILES: [2H]C([2H])(Oc1c(OC)nc(N)nc1OC)C(F)F]

The title compound was prepared in analogy to Intermediate B4 from 5-(1,1-dideuterio-2,2-difluoro-ethoxy)-N,N-bis[(2,4-dimethoxyphenyl) methyl]-4,6-dimethoxy-pyrimidin-2-amine instead of 5-(1,1-dideuterio-2-fluoro-ethoxy)-N,N-bis[(2,4-dimethoxyphenyl)methyl]-4-methoxy-pyrimidin-2-amine in step 3) as off-white solid, MS (ESI): m/z=238.0 [M+H]⁺.

### EXAMPLES

### Example 1: 6-chloro-7-fluoro-N-[4-methoxy-5-(1,1,2,2-tetradeuterio-2-fluoro-ethoxy) pyrimidin-2-yl]-1H-indole-3-sulfonamide

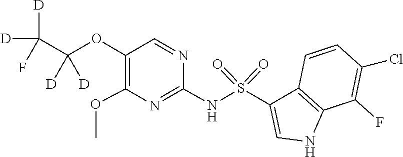 [SMILES: [2H]C([2H])(F)C([2H])([2H])Oc1cnc(NS(=O)(=O)c2c[nH]c3c(F)c(Cl)ccc23)nc1OC]

To a solution of 6-chloro-7-fluoro-1H-indole-3-sulfonyl chloride (337.79 mg, 1.26 mmol, intermediate Al) and [4-methoxy-5-(1,1,2,2-tetradeuterio-2-fluoro-ethoxy) pyrimidin-2-yl]amine (172.08 mg, 900 umol, intermediate B1) in acetonitrile (3.5 mL) was added at 22° C. potassium phosphate tribasic (286.56 mg, 1.35 mmol). The mixture was stirred at 22° C. for 16 h. The mixture was treated with a saturated aqueous solution of sodium bicarbonate (25 mL) and extracted with ethyl acetate (3×25 mL). The organic layers were dried over sodium sulfate, filtered and concentrated in vacuo. The residue was purified by flash chromatography over silica gel using a gradient ethylacetate/heptane 30-100% to provide the title compound as a white solid (190 mg, 50% yield). MS (ESI): m/z=423.1 [M+H]+.

### Example 2: 6-bromo-N-[4,6-dimethoxy-5-(1,1,2,2-tetradeuterio-2-fluoro-ethoxy) pyrimidin-2-yl]-1H-pyrrolo [2,3-b]pyridine-3-sulfonamide

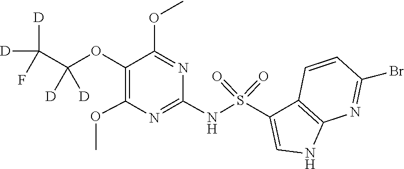 [SMILES: [2H]C([2H])(F)C([2H])([2H])Oc1c(OC)nc(NS(=O)(=O)c2c[nH]c3nc(Br)ccc23)nc1OC]

A solution of [4,6-dimethoxy-5-(1,1,2,2-tetradeuterio-2-fluoro-ethoxy) pyrimidin-2-yl]amine (200 mg, 876.96 umol, intermediate B2) and n-ethyldiisopropylamine (173.48 mg, 229.47 μL, 1.32 mmol) in 1,2-dichloroethane (4 mL) was heated to 70° C. 6-bromo-1H-pyrrolo [2,3-b]pyridine-3-sulfonyl chloride (327.38 mg, 1.05 mmol, intermediate A3) was added in 10 portions over 27 min. The stirring was continued at 70° C. for 15 min. The reaction mixture was poured into water and extracted twice with ethylacetate. The organic layers were dried over sodium sulfate, filtered and concentrated in vacuo. The residue was purified by flash chromatography over silica gel using a gradient ethylacetate/heptane 0-100% to provide the title compound as a white solid (53 mg, 12% yield). MS (ESI): m/z=482.1 [M+H]+.

### Example 3: 6-chloro-N-[4,6-dimethoxy-5-(1,1,2,2-tetradeuterio-2-fluoro-ethoxy)pyrimidin-2-yl]-1H-pyrrolo[2,3-b]pyridine-3-sulfonamide

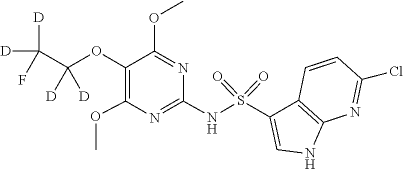 [SMILES: [2H]C([2H])(F)C([2H])([2H])Oc1c(OC)nc(NS(=O)(=O)c2c[nH]c3nc(Cl)ccc23)nc1OC]

To a stirred solution of [4,6-dimethoxy-5-(1,1,2,2-tetradeuterio-2-fluoro-ethoxy) pyrimidin-2-yl]amine (164 mg, 741.34 umol intermediate B2) and n-ethyldiisopropylamine (146.65 mg, 193.99 uL, 1.11 mmol) in dichloromethane (8 mL) was added a suspension of 6-chloro-1H-pyrrolo [2,3-b] pyridine-3-sulfonyl chloride (241.99 mg, 963.75 umol, intermediate A4) in ethyl acetate (8 mL) dropwise at room temperature. The reaction mixture was stirred for 1 hr, poured into water and extracted twice with ethylacetate. The organic layers were dried over sodium sulfate, filtered and concentrated in vacuo. The residue was purified by flash chromatography over silica gel using a gradient ethylacetate/heptane 0-100% to provide the title compound as a white solid (53 mg, 16% yield). MS (ESI): m/z=436.1 [M+H]+

### Example 4: N-[5-(1,1-dideuterio-2,2-difluoro-ethyl)-4-methoxy-pyrimidin-2-yl]-6-fluoro-1H-indole-3-sulfonamide

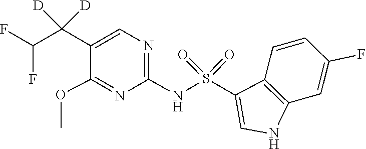 [SMILES: [2H]C([2H])(c1cnc(NS(=O)(=O)c2c[nH]c3cc(F)ccc23)nc1OC)C(F)F]

The title compound was prepared in analogy to example 3 by coupling intermediate A2 with intermediate B3 as a white solid. MS (ESI): m/z=389.2 [M+H]+.

The following Examples 5-8 were prepared in analogy to Example 1 by coupling the indicated sulfonylchloride intermediates A and amine intermediates B.

| | | | | | MS |
|---|---|---|---|---|---|
| | | | | | (ESI): |
| Ex. | Structure | Name | Int. A | Int. B | m/z |
| 5 | 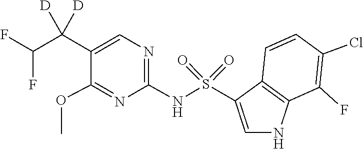 [SMILES: [2H]C([2H])(c1cnc(NS(=O)(=O)c2c[nH]c3c(F)c(Cl)ccc23)nc1OC)C(F)F] | 6-chloro-N-[5-(1,1- dideuterio-2,2-difluoro- ethyl)-4-methoxy- pyrimidin-2-yl]-7-fluoro- 1H-indole-3-sulfonamide | A1 | B3 | 423.2 [M + H]⁺ |
| 6 | 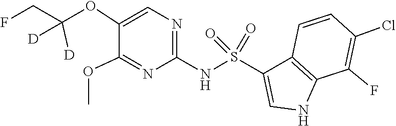 [SMILES: [2H]C([2H])(CF)Oc1cnc(NS(=O)(=O)c2c[nH]c3c(F)c(Cl)ccc23)nc1OC] | 6-chloro-N-[5-(1,1- dideuterio-2-fluoro-ethoxy)- 4-methoxy-pyrimidin-2-yl]- 7-fluoro-1H-indole-3- sulfonamide | A1 | B4 | 421.1 [M + H]⁺ |
| 7 | 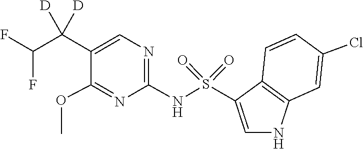 [SMILES: [2H]C([2H])(c1cnc(NS(=O)(=O)c2c[nH]c3cc(Cl)ccc23)nc1OC)C(F)F] | 6-chloro-N-[5-(1,1- dideuterio-2,2-difluoro- ethyl)-4-methoxy- pyrimidin-2-yl]-1H-indole- sulfonamide | A5 | B3 | 405.1 [M + H]⁺ |
| 8 | 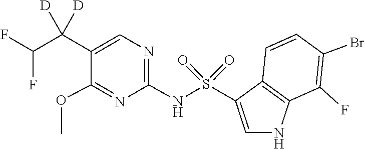 [SMILES: [2H]C([2H])(c1cnc(NS(=O)(=O)c2c[nH]c3c(F)c(Br)ccc23)nc1OC)C(F)F] | 6-bromo-N-[5-(1,1- dideuterio-2,2-difluoro- ethyl)-4-methoxy- pyrimidin-2-yl]-7-fluoro- 1H-indole-3-sulfonamide | A7 | B3 | 469.2 [M + H]⁺ |

The following Examples 9-11 were prepared in analogy to Example 2 by coupling the indicated sulfonylchloride intermediates A and amine intermediates B.

| | | | | | MS |
|---|---|---|---|---|---|
| | | | | | (ESI): |
| Ex. | Structure | Name | Int. A | Int. B | m/z |
| 9 | 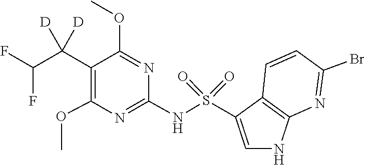 [SMILES: [2H]C([2H])(c1c(OC)nc(NS(=O)(=O)c2c[nH]c3nc(Br)ccc23)nc1OC)C(F)F] | 6-bromo-N-[5-(1,1- dideuterio-2,2-difluoro- ethyl)-4,6-dimethoxy- pyrimidin-2-yl]-1H- pyrrolo[2,3-b]pyridine-3- sulfonamide | A3 | B5 | 482.1 [M + H]⁺ |
| 10 | 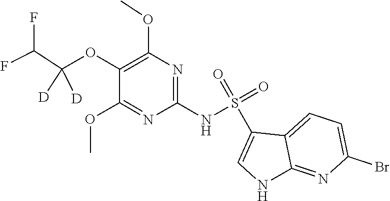 [SMILES: [2H]C([2H])(Oc1c(OC)nc(NS(=O)(=O)c2c[nH]c3nc(Br)ccc23)nc1OC)C(F)F] | 6-bromo-N-[5-(1,1- dideuterio-2,2-difluoro- ethoxy)-4,6-dimethoxy- pyrimidin-2-yl]-1H- pyrrolo[2,3-b]pyridine-3- sulfonamide | A3 | B6 | 498.0 [M + H]⁺ |
| 11 | 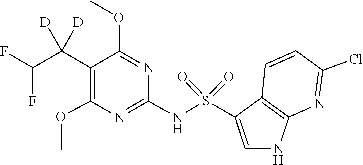 [SMILES: [2H]C([2H])(c1c(OC)nc(NS(=O)(=O)c2c[nH]c3nc(Cl)ccc23)nc1OC)C(F)F] | 6-chloro-N-[5-(1,1- dideuterio-2,2-difluoro- ethyl)-4,6-dimethoxy- pyrimidin-2-yl]-1H- pyrrolo[2,3-b]pyridine-3- sulfanomide | A4 | B5 | 436.1 [M + H]⁺ |

### Example 12: N-[5-(1,1-dideuterio-2,2-difluoro-ethyl)-4,6-dimethoxy-pyrimidin-2-yl]-6-methyl-1H-pyrrolo [2,3-b]pyridine-3-sulfonamide

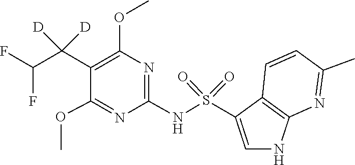 [SMILES: [2H]C([2H])(c1c(OC)nc(NS(=O)(=O)c2c[nH]c3nc(C)ccc23)nc1OC)C(F)F]

The title compound was prepared in analogy to Example 3 by coupling intermediate A6 with intermediate B5 as a white solid. MS (ESI): m/z=414.2 [M-H]⁻.

### Example A

A compound of formula I can be used in a manner known per se as the active ingredient for the production of tablets of the following composition:

| | Per tablet |
|---|---|
| Active ingredient | 200 mg |
| Microcrystalline cellulose | 155 mg |
| Corn starch | 25 mg |
| Talc | 25 mg |
| Hydroxypropylmethylcellulose | 20 mg |
| | 425 mg |

### Example B

A compound of formula I can be used in a manner known per se as the active ingredient for the production of capsules of the following composition:

| | Per capsule |
|---|---|
| Active ingredient | 100.0 mg |
| Corn starch | 20.0 mg |
| Lactose | 95.0 mg |
| Talc | 4.5 mg |
| Magnesium stearate | 0.5 mg |
| | 220.0 mg |

1. A compound of formula I 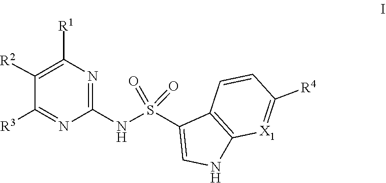
wherein
R¹ is H, alkoxy, or haloalkoxy;
R² is 1,1,2,2-tetradeuterio-2-fluoro-ethoxy, 1,1-dideuterio-2-fluoro-ethoxy, or 1,1-dideuterio-2,2-difluoro-ethyl;
R³ is alkoxy or haloalkoxy;
X₁ is CR⁵ or N;
R⁴ is H, halo, alkyl, or haloalkyl;
R⁵ is H or halo;
and pharmaceutically acceptable salts.

2. The compound of claim 1, wherein R¹ is H or alkoxy.

3. The compound of claim 1, wherein R² is 1,1,2,2-tetradeuterio-2-fluoro-ethoxy, or 1,1-dideuterio-2,2-difluoro-ethyl.

4. The compound of claim 1, wherein R³ is alkoxy.

5. The compound of claim 1, wherein R⁴ is halo or alkyl.

6. The compound of claim 1, wherein R⁴ is halo.

7. The compound of claim 1, wherein R⁵ is halo.

8. The compound of claim 1 wherein
R¹ is H, alkoxy;
R² is 1,1,2,2-tetradeuterio-2-fluoro-ethoxy, 1,1-dideuterio-2-fluoro-ethoxy, or 1,1-dideuterio-2,2-difluoro-ethyl;
R³ is alkoxy;
X₁ is CR⁵ or N;
R⁴ is H, halo;
R⁵ is H or halo;
and pharmaceutically acceptable salts.

9. The compound of claim 1 wherein
R¹ is H, alkoxy;
R² is 1,1,2,2-tetradeuterio-2-fluoro-ethoxy, or 1,1-dideuterio-2,2-difluoro-ethyl;
R³ is alkoxy;
X₁ is CR⁵ or N;
R⁴ is H, halo;
R⁵ is H or halo;
and pharmaceutically acceptable salts.

10. The compound of claim 1, wherein the compound is selected from
6-chloro-7-fluoro-N-[4-methoxy-5-(1,1,2,2-tetradeuterio-2-fluoro-ethoxy)pyrimidin-2-yl]-1H-indole-3-sulfonamide;
6-bromo-N-[4,6-dimethoxy-5-(1,1,2,2-tetradeuterio-2-fluoro-ethoxy)pyrimidin-2-yl]-1H-pyrrolo [2,3-b]pyridine-3-sulfonamide;
6-chloro-N-[4,6-dimethoxy-5-(1,1,2,2-tetradeuterio-2-fluoro-ethoxy) pyrimidin-2-yl]-1H-pyrrolo[2,3-b]pyridine-3-sulfonamide;
N-[5-(1,1-dideuterio-2,2-difluoro-ethyl)-4-methoxy-pyrimidin-2-yl]-6-fluoro-1H-indole-3-sulfonamide;
and pharmaceutically acceptable salts thereof.

11. A The compound of claim 1, wherein the compound is selected from
6-chloro-N-[5-(1,1-dideuterio-2,2-difluoro-ethyl)-4-methoxy-pyrimidin-2-yl]-7-fluoro 1H-indole-3-sulfonamide;
6-chloro-N-[5-(1,1-dideuterio-2-fluoro-ethoxy)-4-methoxy-pyrimidin-2-yl]-7-fluoro-1H-indole-3-sulfonamide;
6-chloro-N-[5-(1,1-dideuterio-2,2-difluoro-ethyl)-4-methoxy-pyrimidin-2-yl]-1H-indole-3-sulfonamide;
6-bromo-N-[5-(1,1-dideuterio-2,2-difluoro-ethyl)-4-methoxy-pyrimidin-2-yl]-7-fluoro-1H-indole-3-sulfonamide;
6-bromo-N-[5-(1,1-dideuterio-2,2-difluoro-ethyl)-4,6-dimethoxy-pyrimidin-2-yl]-1H-pyrrolo [2,3-b]pyridine-3-sulfonamide;
6-bromo-N-[5-(1,1-dideuterio-2,2-difluoro-ethoxy)-4,6-dimethoxy-pyrimidin-2-yl]-1H-pyrrolo [2,3-b]pyridine-3-sulfonamide;
6-chloro-N-[5-(1,1-dideuterio-2,2-difluoro-ethyl)-4,6-dimethoxy-pyrimidin-2-yl]-1H-pyrrolo [2,3-b]pyridine-3-sulfonamide;
N-[5-(1,1-dideuterio-2,2-difluoro-ethyl)-4,6-dimethoxy-pyrimidin-2-yl]-6-methyl-1H-pyrrolo [2,3-b]pyridine-3-sulfonamide;
and pharmaceutically acceptable salts thereof.

12. A process to prepare a compound of claim 1 comprising reacting a compound of formula II 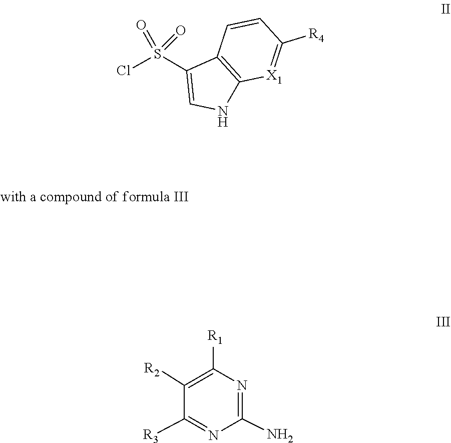
in the presence of a base selected from N,N-diisopropylethylamine, pyridine, potassium phosphate or sodium hydride to provide a compound of formula I 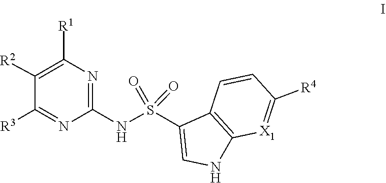
wherein the substituents R¹, R², R³, R⁴ and X₁ are as defined above.

13-14. (canceled)

15. The pharmaceutical composition comprising a of claim 1 and a therapeutically inert carrier.

16-20. (canceled)

21. A method for the treatment or prophylaxis of one or more conditions resulting from direct damage to myelin sheaths, demyelinating disorders, CNS disorders associated with myelin loss, or inflammation in the CNS, which method comprises administering an effective amount of a compound according to claim 1 to a patient in need thereof.

22. The method of claim 21, wherein the one or more conditions comprises multiple sclerosis.

23-24. (canceled)
